(19) United States
(12) Patent Application Publication
(10) Pub. No.: US 2002/0131961 A1
(43) Pub. Date: Sep. 19, 2002

(54) Method for gene transfer using Bcl2 and compositions useful therein

(75) Inventors: James M. Wilson, Gladwyne, PA (US); Shu-Jen Chen, Aldan, PA (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(73) Assignee: The Trustees of the University of Pennsylvania, Philadelphia, PA

(21) Appl. No.: 10/060,762

(22) Filed: Jan. 30, 2002

Related U.S. Application Data
(63) Continuation of application No. 09/528,427, filed on Mar. 17, 2000.
Application No. 09/528,427 is a continuation of application No. PCT/US98/19470, filed on Sep. 18, 1998.
(60) Provisional application No. 60/059,556, filed on Sep. 19, 1997.

(51) Int. Cl. A61K 48/00; A61K 39/235; C12N 15/861

(52) U.S. Cl. 424/93.21; 435/456; 424/233.1

(57) ABSTRACT

A method for liver-directed gene therapy is described. The method involves transfer of Bcl2 and a selected transgene to hepatocytes. Bcl2 protects those hepatocytes which express it from apoptosis and permits proliferation of hepatocytes containing the transgene.

### CROSS-REFERENCE TO RELATED APPLICATIONS

This is a continuation of U.S. patent application Ser. No. 09/528,427, filed Mar. 17, 2000, which is a continuation of International Patent Application No. PCT/US98/19470, filed on Sep. 18, 1998, which claims the benefit of the priority of U.S. Patent Application No. 60/059,556, filed on Sep. 19, 1997, now abandoned.

This work was supported by grants from the National Institutes of Health P30-DK44757-04 and PO1-HD32649-03. The U.S. government has certain rights in this invention.

### BACKGROUND OF THE INVENTION

This invention relates generally to methods for gene transfer, and particularly, to methods for gene transfer using viral vectors.

Adeno-associated virus (AAV), possesses unique features that make it attractive as a vector for delivering foreign DNA to cells. Unlike other viral vectors, AAVs have not been shown to be associated with any known human disease and are generally not considered pathogenic. Wild-type AAV is capable of integrating into host chromosome in a site-specific manner.

However, studies of recombinant AAV (rAAV) in vitro have been disappointing because of low frequencies of transduction; incubation of cells with rAAV in the absence of contaminating wild-type AAV or helper adenovirus is associated with little recombinant gene expression [D. Russell et al, Proc. Natl. Acad. Sci. USA 91:8915-8919 (1994); I. Alexander et al, J. Virol. 68:8282-8287(1994); D. Russell et al, Proc. Natl. Acad. Sci. USA, 92:5719-5723 (1995); K. Fisher et al, J. Virol., 70:520-532 (1996); and F. Ferrari et al, J. Virol. 70:3227-3234 (1996)]. Furthermore, chromosomal integration is inefficient and not directed to chromosome 19 when rep is absent [S. Kumar et al, J. Mol. Biol., 222:45-57 (1991)].

What are needed in the art are methods of overcoming the limitations associated with current methods for rAAV gene transfer.

### BRIEF DESCRIPTION OF THE DRAWINGS

FIG. 1A is a circular map of a plasmid used in the construction of an AAV vector expressing Bcl2 under control of a cytomegalovirus promoter.

FIG. 1B is a circular map of a plasmid used in the construction of an Ad vector expressing Bcl2 under control of an albumin promoter.

FIG. 2 is a circular map of a recombinant AAV containing LDLR and Bcl2 under control of a cytomegalovirus promoter.

FIG. 3 illustrates cell death in hepatocytes infected with the recombinant viruses AdBcl2, AAVBcl2, AAVBcl2+AdLacZ or AdLacZ, following incubation with either tumor necrosis factor or Fas antibody. Percentage of cell death was microscopically determined by DAPI staining of cell nuclei.

FIG. 4 is a graph charting in vivo dose titration of Fas antibody.

FIG. 5 is a graph of the survival rates in mice infused with the recombinant viruses, Ad.AlbBcl2, AAVBcl2, Ad.LacZ+AAVBcl2, and Ad.HGF, followed by Fas antibody.

FIG. 6 illustrates Bcl2 expression in mice receiving AAVBcl2. Clonal expansion of Bcl2 expressing cells was detected in the animals receiving virus followed by Fas antibody, and quantitated.

FIG. 7 is a circular map of a plasmid used in the construction of a recombinant AAV which contains the CB promoter, Bcl2, an IRES, a gene encoding αl-antitrypsin, and a polyA site.

FIG. 8 is a circular map of a plasmid used in the construction of a recombinant AAV which contains the chicken β-actin promoter (CB), an erythropoietin (Epo) gene, an internal ribozyme entry site (IRES), Bcl2, and a polyA site.

### SUMMARY OF THE INVENTION

The present invention provides a method for gene transfer comprising the step of exposing a population of host cells to a recombinant viral vector which comprises a gene encoding an anti-apoptotic agent, a selected transgene, and regulatory sequences which control expression of said anti-apoptotic agent and said transgene. This exposure step permits infection of a subpopulation of the host cells with the recombinant viral vector. The entire population of host cells is then contacted with an apoptotic agent, whereby the subpopulation of infected host cells are protected against apoptosis and survive to proliferate. In this manner, the invention provides for selection of host cells containing transgene.

In another aspect, the present invention provides a method for gene transfer comprising the steps of exposing a population of host cells to a first recombinant viral vector comprising a gene encoding an anti-apoptotic agent and regulatory sequences which control expression thereof, whereby a subpopulation of said host cells are infected with said first recombinant viral vector. The entire population of host cells is also exposed to a second recombinant viral vector comprising a selected transgene and regulatory sequences which control expression thereof, whereby a subpopulation of said host cells are infected with the second recombinant viral vector. The entire population of host cells is then contacted with an apoptotic agent, whereby the subpopulation of host cells infected with the vector containing the anti-apoptotic agent is protected against apoptosis.

In yet another aspect, the present invention provides a recombinant viral vector comprising a Bcl2 gene which is an inhibitor of apoptosis, a selected transgene, and regulatory sequences which direct expression of the Bcl2 gene product and the transgene product. Preferably, the vector integrates into the host chromosome.

In still another aspect, the present invention provides a pharmaceutical composition comprising the recombinant viral vector of the invention and a suitable carrier or delivery vehicle.

Other aspects and advantages of the present invention are described further in the following detailed description of the preferred embodiments thereof.

### DETAILED DESCRIPTION OF THE INVENTION

The present invention provides a method for gene transfer, as well as viral vectors and pharmaceutical compositions useful in the method of the invention. The method of the invention is useful for achieving stable and efficient genetic reconstitution in liver following direct administration of a recombinant viral vector, e.g., rAAV, and selective expansion of transduced cells. The invention is also useful for gene therapy.

Advantageously, the invention overcomes the problems associated with low transduction efficiencies, by selecting for cells expressing the transgene followed by regeneration (i.e., proliferation) of these cells. Further, the method of the invention avoids the necessity to repeatedly administer vectors by permitting their replication during cellular proliferation.

I. Method of the Invention

The invention involves exposing a population of host cells to a recombinant viral vector containing an anti-apoptotic agent and a selected transgene, under conditions which permit infection of a subpopulation of the host cells with the recombinant viral vector. Suitably, the recombinant viral vector, and thus the transgene, replicates upon division of the cells which it transduces and is passed on to the progeny cells. In an alternative embodiment, the present invention permits the anti-apoptotic agent and the selected transgene to be carried on separate recombinant viral vectors.

As used herein, the term “anti-apoptotic agent” refers to any product which is capable of protecting a host cell containing the agent against apoptosis. Preferably, the anti-apoptotic agent utilized in the invention is selected from the anti-apoptotic members of the Bcl2 family of genes. The presently preferred anti-apoptotic agent is Bcl2. The ability of Bcl2 to protect against anti-Fas antibody-induced liver injury has been studied [see, for example, V. Lacronique et al., Nature Med., 2(1):80-86 (January 1996)]. The cDNA sequence of Bcl2 is described in Y. Tsujimoto & C. M. Croce, Proc. Natl. Acad. Sci. USA, 83:5214-5218 (1986). However, the skilled artisan will recognize that other anti-apoptotic members of the Bcl2 family, e.g., Bcl-x₁, can be readily substituted. Alternatively, other inhibitors of interleukin-1β-converting enzyme (ICE)-type proteases and/or inhibitors of apoptosis may be substituted for Bcl2, and the apoptotic agent utilized in the invention adjusted accordingly. For convenience throughout this specification, reference will be made to Bcl2. However, it will be understood from the foregoing that other anti-apoptotic agents may be readily utilized in the method and constructs of the invention.

Following exposure of the host cells to the recombinant viral vector or vectors, the entire population of host cells is contacted with an apoptotic agent, resulting in ablation of host cells not carrying the anti-apoptotic agent. The apoptotic agent used in the method of the invention is selected in conjunction with the choice of protective anti-apoptotic gene. For example, where the method utilizes Bcl2 as the anti-apoptotic agent, the apoptotic agent is preferably selected from among non-neutralizing anti-fas antibodies. However, other suitable apoptotic agents for use in the method of the invention include, without limitation, members of the tumor necrosis factor (TNF) family, and chemical reagents, such as those conventionally used in chemotherapeutic regimens, against which Bcl2 confers protection. Also useful are hydrogen peroxide, free radicals, glucose deprivation, and γ- and UV-radiation, against which Bcl2 also confers protection. Where an alternative to a member of the Bcl2 family is utilized as the anti-apoptotic agent, appropriate apoptotic agents may be readily selected.

Where the host cells contain both the anti-apoptotic agent and the selected transgene, the method of the invention permits selective repopulation of the tissue culture or tissue with transgene-containing cells by protecting these cells with the apoptotic agent. Where the host cells have been exposed to separate vectors containing the anti-apoptotic agent and the selected transgene, the cells which survive exposure to the apoptotic agent include cells uninfected with transgene. Nevertheless, this embodiment provides an increase in the percentage of the cells in the tissue or tissue culture which contain transgene.

As exemplified herein, the method of the invention is particularly well suited for use with liver cells, i.e., hepatocytes, both in vitro and in vivo. For example, where the method of the invention is directed to treatment of the liver, the surviving hepatocytes repopulate the liver, and carry the transgene-expressing rAAV. However, the skilled artisan will recognize that it may also be readily utilized with other cells, and particularly tissue-derived cells with the capacity to regenerate, including lung, muscle, and epithelial cells, among others.

II. Viral Vectors

As stated above, the invention provides a single vector carrying both Bcl2 and the selected transgene under the control of regulatory sequences which control expression thereof However, the method of the invention permits use of separate vectors carrying Bcl2 and the selected transgene.

The transgene useful in the methods and constructs of the invention is a nucleic acid sequence which encodes a product for administration and expression in host cells in vivo or ex vivo to replace or correct an inherited or non-inherited genetic defect or treat an epigenetic disorder or disease. In a particularly preferred embodiment, a transgene for which expression in the liver, i.e., hepatocytes, is desirable is utilized.

Currently preferred transgenes include low density lipoprotein receptor (LDLr), very low density lipoprotein receptor (VLDLr), growth hormone, Factor IX, and liver enzyme genes, such as ornithine transcarbamylase (OTC), carbamyl phosphate synthetase (CPS), arginino-succinate lysase (AL), arginase (ARG), and arginino-succinate synthetase (AS). However, this method is anticipated to be useful with any transgene.

While any viral vector may be utilized in the method of this invention, viral vectors or other vectors which replicate during division of the host cell are most desirable. Suitably, these viral vectors integrate into the host chromosome and are selected from among murine retroviruses, lentiviruses, and hybrid adenovirus/adeno-adeno-associated viruses, such as those described in W096/26286 (Aug. 29, 1996), among others which integrate. Alternatively, vectors which form replicating episomes in the host cells may be utilized, including, without limitation, vectors derived from Epstein-Barr Virus and papilloma virus. Although less desirable, it may be possible to utilize such viral vectors as recombinant poxviruses, recombinant adenoviral vectors, and non-lentivirus retroviral vectors, many of which are known in the art.

The currently preferred vectors for use in the invention, recombinant AAV vectors and recombinant lentivirus vectors are described below. For convenience, the following discussion will be directed to such a vector containing both the Bcl2 and transgene sequences. However, the skilled artisan will understand that using these techniques and those known in the art, a vector may be constructed which contains only the Bcl2 or transgene sequence, in addition to the other vector elements discussed below.

A. AAV Vectors

Many rAAV vectors are known to those of skill in the art and the invention is not limited to any particular rAAV vector. For example, AAV vectors and methods of producing them are described in U.S. Pat. No. 5,252,479; U.S. Pat. No. 5,139,941; International Patent Application No. WO94/13788; and International Patent Application No. WO93/24641. One particularly useful vector is described below.

Currently, a preferred rAAV has all viral open reading frames (ORFs) deleted and retains only the cis-acting 5′ and 3′ inverted terminal repeat (ITR) sequences [See, e.g., B. J. Carter, in “Handbook of Parvoviruses”, ed., P. Tijsser, CRC Press, pp. 155-168 (1990)]. Thus, the rep and cap polypeptide encoding sequences are deleted. The AAV ITR sequences are about 143 bp in length. While it is preferred that substantially the entire 5′ and 3′ sequences which comprise the ITRs are used in the vectors, the skilled artisan will understand that some degree of minor modification of these sequences is permissible. The ability to modify these ITR sequences while retaining their biological functions is within the skill of the art. See, e.g., texts such as Sambrook et al, “Molecular Cloning. A Laboratory Manual. ”, 2d edit., Cold Spring Harbor Laboratory, New York (1989).

The AAV ITR sequences may be obtained from any known AAV, including presently identified human AAV types. The selection of the AAV type does not limit the invention. A variety of AAV types, including types 1-4, are available from the American Type Culture Collection or are available by request from a variety of commercial and institutional sources. Similarly, AAVs known to infect other animals may also be employed in the vector used in the methods of this invention.

In addition to the AAV ITR sequences, the Bcl2 sequences, and the transgene, the vector also includes regulatory elements necessary to drive expression of Bcl2 and the transgene product in the infected host cells. Thus the vector desirably contains a selected promoter and enhancer (if desired), operatively linked to Bcl2 and the transgene and located, with Bcl2 and the transgene, between the AAV ITR sequences of the vector.

Selection of the promoter and, if desired, the enhancer, is a routine matter and is not a limitation of the vector itself Useful promoters may be constitutive promoters or regulated (inducible) promoters, which will enable controlled expression of the transgene. For example, a desirable promoter is the liver specific albumin promoter. Another desirable promoter is a β-actin promoter, which is desirably used in combination with a cytomegalovirus (CMV) enhancer. Still other desirable promoters include, without limitation, the Rous sarcoma virus LTR promoter/enhancer, the cytomegalovirus immediate early promoter/enhancer [see, e.g., Boshart et al, Cell, 41:521-530 (1985)], and the inducible mouse metallothienien promoter. Still other promoter/enhancer sequences may be selected by one of skill in the art.

The vectors will also desirably contain nucleic acid sequences which maximize efficient transcription or translation of the anti-apoptotic agent (e.g., Bcl2) and transgene, including sequences providing signals required for efficient polyadenylation of the transcript, introns with functional splice donor and acceptor sites, and internal ribozyme entry sites (IRES) A common poly-A sequence is that derived from the papovavirus SV-40. The poly-A sequence generally is inserted into the vector following the transgene and Bcl2 sequences and before the 3′ AAV ITR sequence. A common intron sequence is also derived from SV-40, and is referred to as the SV-40 T intron sequence. Selection of these and other elements desirable to control or enhance gene expression are conventional and many such sequences are known to those of skill in the art [see, e.g., Sambrook et al, and references cited therein].

B. Lentivirus Vectors

Suitable lentiviral vectors are well known to those of skill in the art. See, e.g., WO 95/25806 (Sep. 28, 1995). The recombinant feline immunodeficiency virus (FIV) contains Bcl2 and a selected transgene for delivery to a cell and a heterologous envelope protein which provides a pseudotype of broad tropism.

The construction of one desirable rFIV vector of the invention involves novel modifications of known methods for production of HIV vectors. See, e.g., Naldini et al., Science 272:263-267 (April 1996). The function of the native env protein of the recombinant FIV of the invention is destroyed, either by complete or partial deletion or disruption by other means, e.g., frame shift mutation. The rFIV is provided with a heterologous env protein which is capable of targeting non-feline mammalian cells and, desirably, human cellular receptors. Desirably, the heterologous env protein utilized is the vesicular stomatitis virus G envelope protein, which confers broad tropism. Alternatively, one of skill in the art can readily select other appropriate env proteins or other proteins which facilitate cell entry. Such proteins include, e.g., single chain antibodies, ligands to cellular receptors, and envelope proteins from other lentiviruses, e.g., SIV. Although less desirable, envelope proteins derived from other retroviruses, such as gp160 or gp120, or a portion thereof, derived from Human Immunodeficiency Virus (HIV)-1 or HIV-2 may be utilized.

Currently, the preferred FIV strain is NCSCU₁, [ATCC VR2333]. Another suitable FIV strain, Petaluma, is available from the ATCC [ATCC VR- 1312]. However, other FIV strains may isolated using known techniques, or obtained from other sources, and utilized in the construction of recombinant FIV vectors of the invention.

The rFIV vector also includes regulatory elements necessary to drive expression of the transgene in the infected host cells. Thus the vector desirably contains a selected promoter, and enhancer (if desired), which are operatively linked to the transgene. Selection of the promoter and, if desired, the enhancer, is a routine matter and is not a limitation of the vector itself The vectors will also desirably contain nucleic acid sequences which affect transcription or translation of the transgene. Useful promoters, transcription and translation sequences are discussed above in the discussion of rAAV vectors.

In addition to the transgene for delivery to the target cells, its regulatory sequences, and the heterologous env protein, the recombinant virus comprises retroviral 5′ and 3′ LTR sequences which desirably flank the transgene and its regulatory sequences, a gag sequence and a pol sequence. Currently, in a preferred embodiment, the LTR sequences, gag, and pol are of FIV origin. However, the LTR sequences may be derived from other retroviruses, e.g., HIV. Similarly, the gag and pol utilized in the recombinant FIV of the invention may be derived from another source. Other viruses which may supply the LTR sequences, and/or the gag and pol sequences include, e.g., Mason Pfizer Monkey Virus (MPMV), Mouse Mammary Tumour Virus (MMTV), maloney murine leukemia virus, Squirrel Monkey Retrovirus (SMRV), simian immunodeficiency virus, bovine immunodeficiency virus, equine infectious anemia virus and the like.

C. Construction of Viral Vectors

The sequences employed in the construction of the recombinant vectors of this invention may be obtained from commercial or academic sources based on previously published and described materials. These materials may also be obtained from an individual human or veterinary patient or may be generated and selected using standard recombinant molecular cloning techniques known and practiced by those skilled in the art. Any modification of existing nucleic acid sequences used in the production of the recombinant vectors, including sequence deletions, insertions, and other mutations may also be generated using standard techniques.

Assembly of the recombinant vector, including the sequences of recombinant vector, the transgene and other vector elements, may be accomplished using known techniques. Suitable techniques include cDNA cloning such as those described in texts [Sambrook et al, cited above], use of overlapping oligonucleotide sequences of the recombinant viral genome, polymerase chain reaction, and any suitable method which provides the desired nucleotide sequence. Where appropriate, standard transfection and co-transfection techniques are employed to propagate the recombinant viral viruses, and may be readily selected by the skilled artisan. For example, E1-deleted adenoviruses may be employed to propagated rAAV viruses using CaPO₄ transfection. Other conventional methods which may be employed in this invention include homologous recombination of plasmid genomes, plaquing of viruses in agar overlay, methods of measuring signal generation, and the like.

Desirably, the recombinant vectors are purified using conventional means. For example, rAAV may be purified to remove any contaminating adenovirus or wild-type AAV using the methods described in K. J. Fisher et al, J. Virol., 70(1)520-532 (January, 1996), which is incorporated by reference. One of skill in the art can readily select other appropriate purification means.

III. Pharmaceutical Compositions

Desirably, the recombinant vectors utilized in the method of the invention, which are capable of delivering Bcl2 and the selected transgene in a form suitable for expression, are suspended in a biologically compatible solution or pharmaceutically acceptable carrier. Currently, preferred carriers include sterile saline and phosphate buffered saline. However, other aqueous and non-aqueous isotonic sterile injection solutions and aqueous and non-aqueous sterile suspensions known to be pharmaceutically acceptable carriers may be employed for this purpose and are well known to those of skill in the art. Selection of the carrier is not a limiting factor for the present invention.

Optionally, conventional components, such as preservatives, stabilizers, and the like, may be included in the pharmaceutical compositions of the invention. Additionally, it may be desirable to include other active ingredients, which are conventional for treatment of the patient's condition, in the pharmaceutical compositions of the invention.

IV. Delivery of Transgene

The method of the invention may be performed in vitro or in vivo. When used to deliver genes to a mammalian patient, the vectors of this invention are administered in sufficient amounts to provide sufficient levels of cellular transduction that a desired level of gene expression may be obtained. In a preferred embodiment, the vectors or pharmaceutical compositions of the invention are administered intravenously. However, other suitable methods of administration may be selected by one of skill in the art and include, without limitation, intraarterial, intraperitoneal, and intramuscular administration, including site-directed injection.

Although less preferred, the method of the invention may involve ex vivo gene transfer to hepatocytes or other selected host cells or tissues, treatment of the cells with an apoptotic agent, and re-introduction of the cells into a patient.

Dosages of the viral vectors will depend primarily on its purpose for gene delivery, the cell type, such factors as the selected transgene, and the age, weight and health of the patient, and may thus vary. A therapeutically effective dose of the recombinant viral vectors utilized in the present invention is believed to be in the range of from about 1 to about 50 ml of saline solution containing concentrations of from about 1×10⁸ to 1×10¹³ particle forming units (pfu) of vector. Where rAAV is utilized, each dose desirably contains at least 10⁹ pfu rAAV, and more preferably at least 2×10¹⁰ pfu. Where rFIV is utilized, each dose desirably contains 1×10⁸ to 1×10⁹, and preferably about 2×10⁸, particle forming units (pfu). A more preferred human dosage is about 1-20 ml saline solution at the above concentrations.

The levels of expression of the delivered genes can be monitored to determine the selection, adjustment or frequency of administration. Administration of the vectors may be repeated as needed. Preferably, where the method of the invention utilizes separate vectors, the vectors are administered substantially concurrently. However, one of skill in the art may administer the vectors at substantially different times, where desired.

Optionally, the vectors of the invention may be administered in conjunction with other therapies. Alternatively, the vectors of the invention may be administered in conjunction with immune modulators, particularly immunosuppressants. Examples of suitable immune modulators and methods for their administration have been described in WO 96/26285, published Aug. 29, 1996, which is incorporated by reference for the description thereof.

V. Administration of Apoptotic Agent

As discussed above, according to the present invention, the selected apoptotic agent is administered to the patient or added to the cells in vitro, such that the cells expressing Bcl2 are protected against apoptosis and proliferate to repopulate the organ or culture. Administration of the apoptotic agent may be by any appropriate route. However, for in vivo use, intravenous administration is preferred.

Where anti-fas antibodies are utilized in the method of the invention, they are desirably administered in a dose consisting of about 1 mg to about 50 mg, and preferably about 20 mg antibody for an 80 kg mammal. Suitable doses of other apoptotic agents may be readily determined by one of skill in the art based on knowledge of suitable chemotherapeutic doses.

The following examples illustrate the preferred methods and compositions of the invention, but do not limit the scope of the invention.

### EXAMPLE 1

### Construction of a Recombinant AAV Expressing Bcl2

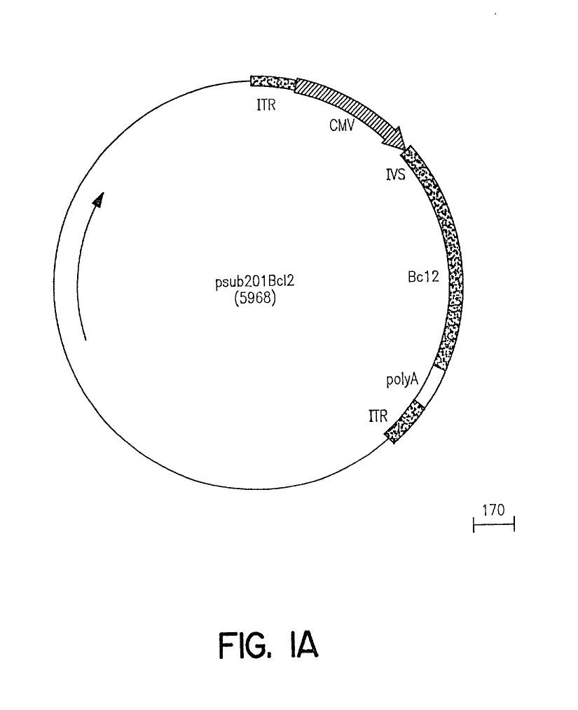

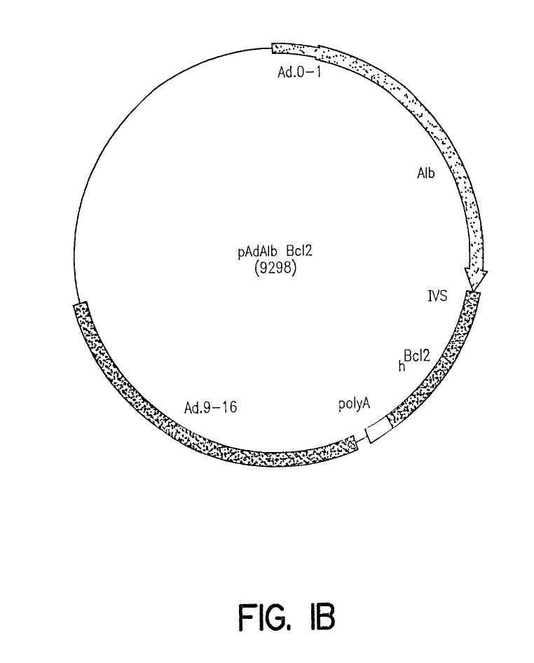

A recombinant AAV virus was prepared by conventional genetic engineering techniques for the purposes of this experiment. Recombinant AAV was generated by plasmid transfections in the presence of helper adenovirus [Samulski et al, J. Virol., 63:3822-3828 (1989)]. The cis-acting plasmid pAV.CMVBcl2 was derived from psub201 [Samulski et al, J. Virol., 61:3096-3101 (1987)] and contains a Bcl2 minigene in place of AAV Rep and Cap genes. See, FIG. 1A. Therefore, the 5′ to 3′ organization of the recombinant AAV.CMVBcl2 genome (5.9 kb) includes

(a) the 5′ AAV ITR (bp 1-173) was obtained by PCR using pAV2 [C. A. Laughlin et al, Gene, 23: 65-73 (1983)] as template;

(b) a CMV immediate early enhancer/promoter [Boshart et al, Cell, 41:521-530 (1985)];

(c) an SV40 intron;

(d) Bcl2 cDNA [nucleotides 1410-2340 of the sequences described in Y. Tsujimoto & C. M. Croce, Proc. Natl. Acad. Sci. USA, 83:5214-5218 (1986)];

(e) an SV40 polyadenylation signal (a 237 Bam HI-BclI restriction fragment containing the cleavage/poly-A signals from both the early and late transcription units); and

(f) 3′ AAV ITR, obtained from pAV2 as a SnaBI-BglII fragment.

Rep and Cap genes were provided by a trans-acting plasmid pAAV/Ad [Samulski et al, cited above].

Monolayers of 293 cells grown to 90% confluency in 150 mm culture dishes were infected with H5.CBALP at an MOI of 10. H5.CBALP is a recombinant adenovirus that contains an alkaline phosphatase minigene in place of adenovirus E1A and E1B gene sequences (map units 1-9.2 of the Ad5 sequence of GenBank [Accession No. M73260]). The alkaline phosphatase cDNA is under the transcriptional control of a CMV-enhanced β-actin promoter in this virus.

Infections were done in Dulbecco's Modified Eagles Media (DMEM) supplemented with 2% fetal bovine serum (FBS) at 20 ml media/150 mm plate. Two hours post-infection, 50 μg plasmid DNA (37.5 μg trans-acting and 12.5 μg cis-acting) in 2.5 ml of transfection cocktail was added to each plate and evenly distributed. Transfections were calcium phosphate based as described [B. Cullen, Meth. Enzymol, 152:684-704 (1987)]. Cells were left in this condition for 10-14 hours after which the infection/transfection media was replaced with 20 ml fresh DMEM/2% FBS. Forty to fifty hours post-transfection, cells were harvested, suspended in 10 mM Tris-Cl (pH 8.0) buffer (0.5 ml/150 mm plate) and a lysate prepared by sonication. The lysate was incubated, after which bovine pancreatic DNase I (20,000 units) and RNase (0.2 mg/ml final concentration) were added, and the reaction incubated at 37° C. for 30 minutes. Sodium deoxycholate was added to a final concentration of 1% and incubated at 37° C. for an additional 10 minutes.

The treated lysate was chilled on ice for 10 minutes and solid CsCl added to a final density of 1.3 g/ml. The lysate was brought to a final volume of 60 ml with 1.3 g/ml CsCl solution in 10 mM Tris-Cl (pH 8.0) and divided into three equal aliquots. Each 20 ml sample was layered onto a CsCl step gradient composed of two 9.0 ml tiers with densities 1.45 g/ml and 1.60 g/ml.

Centrifugation was performed at 25,000 rpm in a Beckman SW-28 rotor for 24 hours at 4° C. One ml fractions were collected from the bottom of the tube and analyzed on 293 or 293(E4) cells for Bcl2 transduction. Fractions containing peak titers of functional AAVCMVBcl2 virus were combined and subjected to three sequential rounds of equilibrium sedimentation in CsCl. Rotor selection included a Beckman Ti70-1 (65,000 rpm for 24 hours) and SW-41 (35,000 rpm for 20 hours). At equilibrium, AAVCMVBcl2 appeared as an opalescent band at 1.40-1.41 g/ml CsCl. Densities were calculated from refractive index measurements. Purified vector was exchanged to 20 mM HEPES buffer (pH7.8) containing 150 mM NaCl (HBS) by dialysis and stored frozen at −80° C. in the presence of 10% glycerol or as a liquid stock at −20° C. in HBS/40% glycerol.

Purified virus was tested for contaminating helper virus and AVCMVBcl2 titers. Helper virus was monitored by histochemical staining for reporter alkaline phosphatase activity. A sample of purified virus representing 1.0% of the final product was added to a growing monolayer of 293 cells seeded in a 60 mm plate. Forty-eight hours later, cells were fixed in 0.5% glutaraldehyde/phosphate buffered saline (PBS) for 10 minutes at room temperature, washed in PBS (3×10 minutes) and incubated at 65° C. for 40 minutes to inactivate endogenous alkaline phosphatase activity. The monolayer was allowed to cool to room temperature, rinsed once briefly in 100 mM Tris-Cl (pH9.5)/100 mM NaCl/5 mM MgCl, and incubated at 37° C. for 30 minutes in the same buffer containing 0.33 mg/ml nitroblue tetrazolium chloride (NBT) and 0.165 mg/ml 5-bromo-4-chloro-3-indolylphosphate p-toluidine salt (BCIP). Color development was stopped by washing the monolayer in 10 mM Tris-Cl (pH 8.0)/5 mM EDTA. Routinely the purification scheme described above removed all detectable H5.CBALP helper virus by the third round of buoyant density ultracentrifugation. Virus particle concentrations were based on Southern blotting.

### EXAMPLE 2

### Construction of Recombinant Adenovirus Expressing Bcl2

As illustrated in FIG. 1B, a recombinant adenovirus expressing Bcl2 was constructed using conventional techniques.

Mouse albumin promoter and enhancer sequence was removed from pAlb(c/p)muPA-GH [J. L. Heckel et al, Cell, 62:447] and human Bcl2 cDNA were subcloned into pAdLinkl [X. Ye et al, J. Biol. Chem., 271:3639-3646 (1996)]. The resulting plasmid, pAdAlbBcl2, contains (from the top in clockwise order) adenovirus sequence map units 0-1; an albumin promoter; intervening sequence (IVS), Bcl2 cDNA, an SV40 polyadenylation signal, adenovirus sequence from map units 9-16 (clear bar), and a portion of the derivative plasmid pAT153 [ATCC No. 57294]. See, FIG. 1B.

Recombinant virus was generated using homologous recombination between pAdAlbBcl2 and Ad5sub360 [J. Logan et al, Proc. Natl. Acad. Sci. USA, 81:3655-3659 (184)] in 293 cells [ATCC CRL1573] using a standard calcium phosphate transfection procedure [see, e.g., Sambrook et al, cited above]. The end result of homologous recombination involving sequences that map to adenovirus map units 9-16.1 is AdAlbBcl2sub360 in which the E1a and E1b coding regions from the dl7001 adenovirus substrate are replaced with the AdAlbBcl2 from the plasmid.

### EXAMPLE 3

### Construction of rAAV Expressing Bcl2 and Transgene

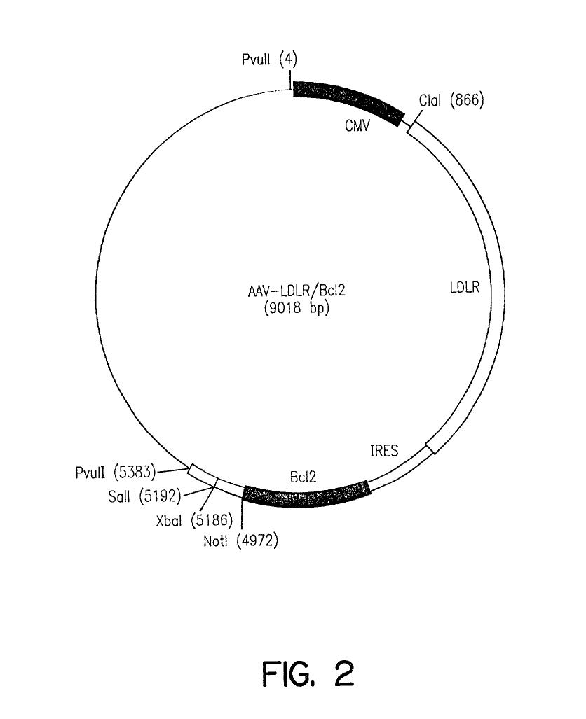

A 0.83 Kb Bcl2 cDNA retrieved from pIB4 [ATCC] with EcoRI and NsiI is subcloned to pCMVLacZ [Promega] to replace the NotI fragment of the LacZ gene. The resulting plasmid is pCMVBcl2. A 1 kb BglII/HindIII fragment which consists of Bcl2 and a polyadenylation signal is excised from pCMVBcl2 and subcloned to pIRES1neo [Clontech] to replace a Smal and XhoI fragment of the Neo gene. LDLR cDNA was obtained by digestion of pLDLR3 [ATCC] with HindIII and SmaI is subcloned to the construct described above to replace the EcoRV and NsiI (IVS) fragment. The bicistronic transcription cassette is excised with NruI and SalI digestion and cloned into psub201 [R. J. Samulski et al, J. Virol., 61:3096-3101 (1987)] in between the two XbaI sites in conjunction with two viral ITRs to generate AAV-Bcl2LDLR, which is illustrated in FIG. 2.

### EXAMPLE 4

### Protection Against Apoptosis In Vitro

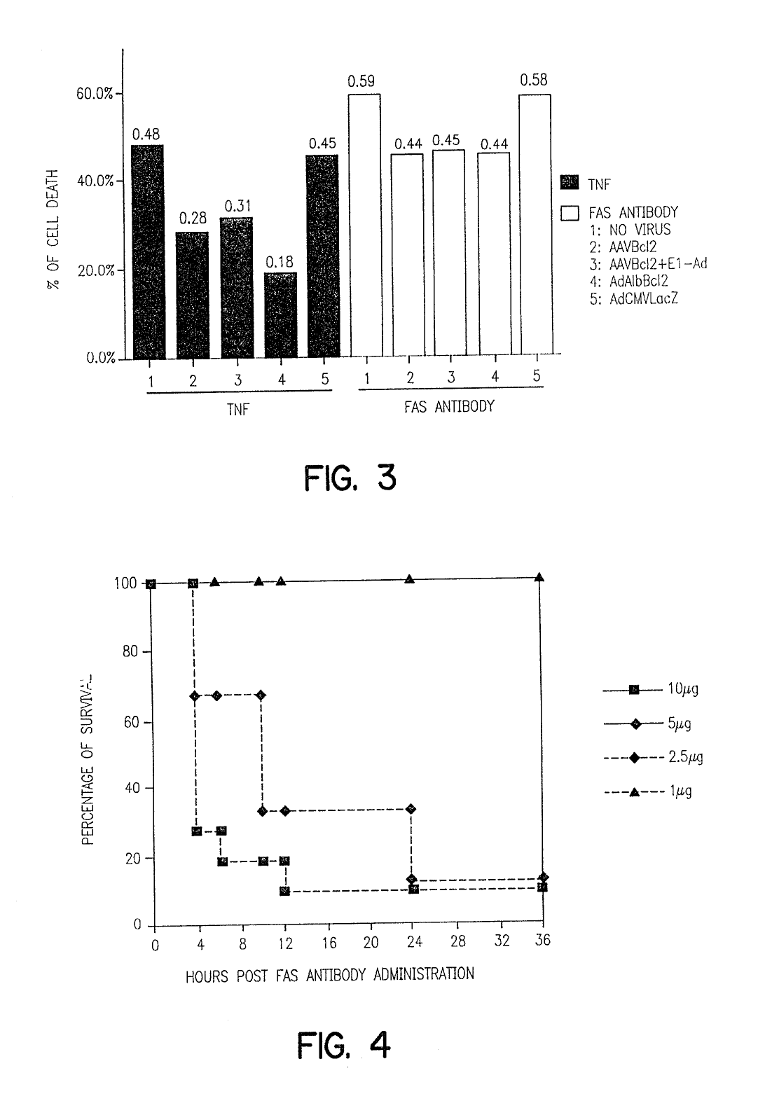

Mouse hepatocytes were infected with AdBcl2, AAVBcl2, AAVBcl2+AdLacZ and AdLacZ, prepared as described in the preceding example. The cells were infected with the recombinant adenoviruses at a moi of 2 and 5 and the recombinant adeno-associated viruses at 1000-10,000 copies of genome/cell on day 2 and incubated at 37° C. for 24 hours. Mouse hepatocytes were treated with mTNF-α (R&D systems, cat#410-MT/CF) at 40 ng/ml plus actinomycin D at 0.5 μg/ml or murine Fas antibody (Jo2 clone, Pharmagen, cat#15400D) at 1 μg/ml plus cycohexamide at 50 μg/mL on day 3 and incubated at 37° C. Following incubation with either tumor necrosis factor or Fas antibody, percentage of cell death was microscopically determined by 4′, 6-diamidino-2-phenylindole (DAPI) staining of cell nuclei as described [C. Jeppesin and P. E. Nielsen, Eur. J. Biochem., 182(2):437-444 (1989)]. The results are illustrated in FIG. 3 The results show that hepatocytes infected with AdBcl2 and AAVBcl2 have a significantly lower percentage of apoptosis compared to cells infected with control virus.

### EXAMPLE 5

### In Vivo Titration of Fas Antibody

Survival was charted in mice receiving 10 μg, 5 μg, 2.5 μg, and 1 μg Fas antibody. The results are provided in FIG. 4.

### EXAMPLE 6

### In Vivo Protection Against Apoptosis

A mouse was infused with 2×1¹⁰ copies of rAAVCMVBcl2 and 1×10¹⁰ particles of AdCMVLacZ via splenic injection and sacrificed on Day 4. High levels of Bcl2 expression were detected in liver by immunofluorescence staining.

In a separate experiment, mice were infused with AdAlbBcl2, AAVBcl2, AdLacZ+AAVBcl2, or a recombinant adenoviral vector containing human growth factor (AdHGF). 1×10¹¹ particles recombinant adenovirus and 2×10¹⁰ copies of recombinant AAV genome were infused via splenic injection as indicated. Fas antibody (5μg, Jo2 clone) was administered on day 3 post-adenovirus infusion and on day 28 post-AAV infusion.

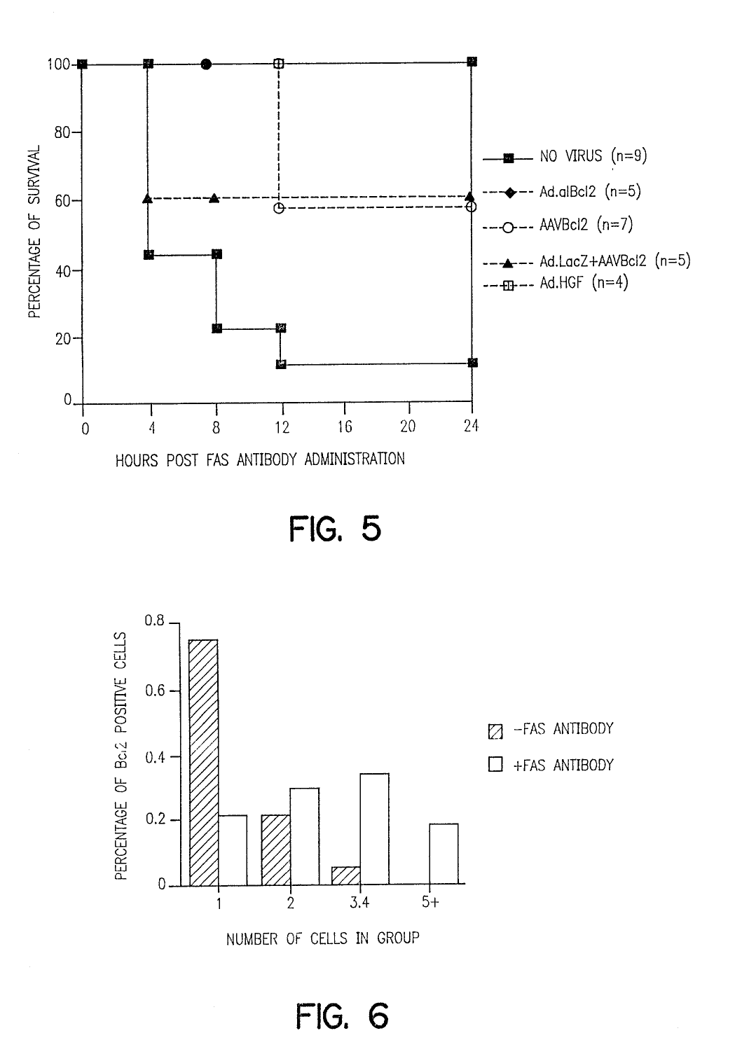

Tissue samples were obtained and subjected to hematoxylin/eosin staining and TUNEL staining. TUNEL staining to detect apoptotic cells in the lever section revealed apoptotic cells in AdBcl2 infused animals at an early time point post-Fas antibody administration. However, the cells were no longer detected at a later time point. Most of the control mice receiving no virus or LacZ virus died within 6 hours post-antibody infusion. Thus, infusion of AdBcl2 and AAVBcl2 is effective in saving animals from i.v. injection of Fas antibody induced animal death. See, FIG. 5.

Bcl2 expression in mice receiving AAVBcl2 was detected. Clonal expansion of Bcl2 expressing cells was detected in animal receiving virus followed by Fas antibody and quantitated. See, FIG. 6. These results indicate that infected cells can tolerate the apoptotic stimuli of Fas antibody and proliferate in response to this injured liver state. Expression of AAV.Bcl2 was also confirmed by Southern blotting and Western blotting, in which the persistence of AAVBcl2 genome was detected in hepatocytes and an increased expression of human Bcl2 protein was detected in liver.

### EXAMPLE 7

### Repopulation of Liver with AAV Transduced Hepatocytes

The following example illustrates that the method of the invention selectively repopulates the liver with vector transduced hepatocytes. As illustrated below, low level, stable transduction of hepatocytes was achieved by direct injection of rAAV into mouse liver. Expansion of these vector transduced cells was achieved by incorporating into the construct a minigene expressing Bcl2 followed by induction of apoptosis in non-vector containing hepatocytes by systemic administration of a Fas antibody. The percent of vector transduced cells increased from 2% to 20% following three administrations of Fas Ab.

A. Production of rAAV Encoding Bcl2

A rAAV encoding Bcl2 was prepared essentially as described in K. J. Fisher et al, J. Virol. 70:520-532 (1996). The human Bcl2 cDNA, a 1 kb fragment, was received from pB4 [Y. Tsujimoto & C. M. Croce, Proc. Natl. Acad, Sci., 83:5214-5218 (1986)] by EcoRI digestion and subcloned to pAlb-uPA [J. L. Heckel et al, Cell, 62:447-456 (1990)] to replace the KpnI/EcoRI fragment encoding uPA to generate pAlb-Bcl2. The Bcl2 cDNA with the murine albumin promoter and polyA signal was removed from pAlb-Bcl2 and subcloned to pSub201 [Fisher et al, cited above] to substitute the XbaI fragment and flanked by two ITRs.

B. Virus Infection of Mouse and Induction of Apoptosis

Recombinant AAV viruses expressing Bcl2 from a liver specific promoter (albumin), prepared as described above, was injected directly into the liver of 6-8 week old immune-deficient Rag^{-/-} mice at a dose equivalent to 2×10¹⁰ copies of AAV genomes. Genetically immune deficient mice were used in these experiments to avoid immunological responses to the human Bcl2 product and to the Fas antibody, which was derived from hamsters. Virus resuspended in HEBs was injected directly into two of the large Rag^{-/-} anterior lobes of the liver (50 μl/lobe). The mice were subsequently given one to three sub-lethal doses (5-10 μg) of agonistic Fas Ab (Jo2 clone from Pharmingen) which were administered intravenously.

The following dosing regimens were used: control—no Fas Ab; group 1—Fas Ab (10 μg) at 5 weeks; group 2—Fas Ab (5 μg) at 4 and 5 weeks; and group 3—5 μg of Fas Ab at 4 and 5 weeks and 10 μg at 6 weeks. All animals were analyzed 8 weeks after gene transfer for expression of Bcl2 in hepatocytes as well as for evidence of liver pathology, using the methods described below.

C. Histochemical Studies

Mouse liver was harvested and embedded in cryopreservative OCT compound (Tissue-Tek). Sections of liver (6 μm) were cut, fixed in cold acetone and subsequently subjected to indirect immunofluorescence staining using rabbit anti-human Bcl2 antiserum (Pharmingen) and secondary FITC-conjugated goat anti-rabbit IgG antibody (Jackson Immuno Research). Paraffin embedded sections were stained with hematoxylin and eosin for analysis of histopathology. Sections were also stained for reticulin as well as with trichrome for collagen.

D. Western Blot

Liver tissue was homogenized with a polytron in Tris buffer (pH 8.) And 150 mM NaCl containing mixtures of protease inhibitors (1 mM phenylmethylsulfonyl fluoride, 1 μg/ml each of leupeptin, antipain chymostatin and soybean trypsin inhibitor). This suspension was subjected to ultracentrifugation at 40,000 rpm at 4° C. for 1 hr. The pellet was reconstituted with the buffer described above and resuspended by passing through 16 and 20 gauge needles 10× each. NP-40 was added to a final concentration of 0.1%. The suspension was incubated on ice for 1 hr and centrifuged. The supernatant was harvested and protein concentration was determined by Lowry assay. Protein (50 μg) was resolved by SDS-PAGE and electrophoretically transferred onto a PVDF membrane (Millipore). Western blotting was performed with monoclonal Bcl2 Ab (DAKO), horseradish peroxidase conjugated mouse IgG Ab (Jackson Immuno Research) and the Enhanced Chemiluminescence (ECL) Western Blotting Detection reagents (Amersham).

E. Results

Multiple section of liver from 2 animals of each group were analyzed for Bcl2 expressing cells. A total of 4 high power fields were analyzed. The mean±standard deviation (SD) is shown. These results are illustrated in Table 1.

TABLE 1

| Groups of Animals | Control | 1 | 2 | 3 | 4 |
|---|---|---|---|---|---|
| Infusion of | − | + | + | + | + |
| AAV-Bcl2 | | | | | |
| Doses of Fas | − | − | 10 μg × 1 | 5 μg × 2 | 5 μg × 2 |
| Antibody | | | | | & |
| | | | | | 10 μg × 1 |
| % of Bcl2 | 0 | 2.22 ± | 6.53 ± | 4.72 ± | 20.13 ± |
| expressing cells | | 0.04 | 1.25 | 0.07 | 4.03 |

Intravenous (data not shown) or intrahepatic (Table 1) administration of AAV Bcl2 was associated with low level transduction that was stable for at least two months (i.e., 2% of hepatocytes were Bcl2 positive). Administration of 10 μg of Fas Ab in one dose (group 1) or two doses (group 2) increased the frequency of Bcl2 cells by 2-3 fold while administration of 20 μg of Fas Ab over 3 doses increased the number of transgene expressing cells 10-fold over baseline to a level of 20% hepatocytes. Western blot analysis of liver homogenates confirmed the proportional increase in Bcl2 expression as a function of Fas Ab treatment. The distribution of transgene expressing cells is most consistent with clonal expansion of individual vector transduced cells. For example, before selection there were scattered transgene expressing cells found in isolation or as doublets. After selection these evolved to clusters of transgene expressing cells ranging in size from 2 to 32 cells in which the intensity of Bcl2 expression varied between clusters but was usually consistent within a cluster.

Microscopic analysis of liver harvested within 24 hours of Fas Ab administration revealed substantial hepatocellular degeneration with multiple apoptotic and mitotic figures (data not shown). The liver returned to essentially normal histology within 10 to 14 days of each antibody administration. The liver histology following vector alone was normal except for sparse focal lymphocytic infiltrates. Overall architecture of the liver was essentially normal following vector and three Fas Ab administrations except for focal lesions characterized by disorganization of the hepatic plates with early regenerative nodules and inflammation. In addition, there was increased reticulin within mid-zonal regions and collagen that extended from central veins into the surrounding parenchyma.

### EXAMPLE 8

### Transduction of Cells with rAAV Co-expressing Transgene and Bcl2

The following example illustrates the ability of exemplary rAAV carrying Bcl2 and selected transgenes to transduce hepatocytes and co-express Bcl2 and the selected transgenes, both in vitro and in vivo.

A. rAAV Expressing Bcl2 and α1-antitrypsin

Plasmid AAV-CB-BA, illustrated in FIG. 7, was generated as follows.

To obtain plasmid pAAVCBAAT, the fragment containing the chicken β-actin promoter with CMV enhancer (CB promoter) was isolated from pAd.CB.hOTC with PstI-NotI [X. Ye et al, J. Biol. Chem., 271:3639-3646 (1996)]. The blunted CB promoter was then cloned into PCI-hAAT at the XbaI site. The PCI-hAAT plasmid had previously been generated by blunting the EcoRI fragment of pAT85 (ATCC) containing α1-antitrypsin cDNA fragment and cloning into PCI (Promega) at a SmaI site. The CB-hAAT expression cassette was removed from PCI-hAAT by NheI and ClaI and cloned into pSub201 at the XbaI site.

Bcl2 cDNA was retrieved as the EcoRI/Nsil fragment of pIB4 [ATCC] and an internal ribozyme entry site (IRES) was retrieved from pIRESlneo [Clontech]. The Bcl2 cDNA and IRES were cloned into pAAVCBAAT upstream of the α1-antitrypsin gene to generate the AAV-CB-BA plasmid. See FIG. 7.

The pAAV-CB-BA plasmid was tested in vitro by transient transfection of 293 cells. Immunofluorescence staining and ELISA with conditioned media confirmed both Bcl2 expression and secretion of αl-antitrypsin by transfected cells.

A rAAV containing both Bcl2 and the gene encoding α1-antitrypsin (AAT) was prepared as described herein [see Example 7] using the AAV-CB-BA plasmid. The resulting rAAV construct contains the AAV ITRs flanking the chicken β-actin promoter, the Bcl2 gene, IRES, AAT, and a polyA sequence.

B. rAAV Expressing Bcl2 and Erythropoietin

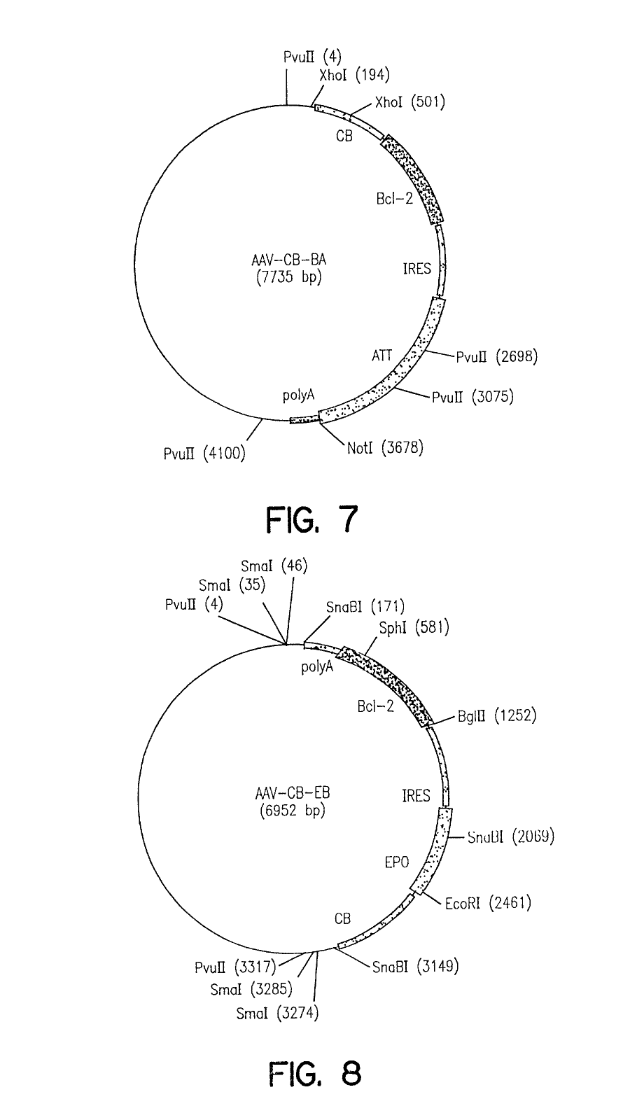

Plasmid AAV-CB-EB, illustrated in FIG. 8, was generated as follows. The Neo gene in pIRESneo was replaced by Bcl2 and the CMV promoter and the intron region was replaced by the Epo gene to generate pIRES Epo/Bcl2. The Epo gene had been previously retrieved as the HindIII/CalI fragment of pZE2. The NhrI/XhoI fragment was retrieved from pIRES Epo/Bcl2 and contains Epo, IRES and Bcl2. This fragment was subcloned into pAAVCBAAT, described above, and replaced the fragment containing α1-antitrypsin, which had been excised following digestion with SalI and NotI to generate pAAV-CB-EB. See FIG. 7. A rAAV containing Bcl2 and Epo were prepared as described in Example 7 [see Fisher et al, cited above] using this plasmid. The rAAV construct contains the AAV ITRs flanking the chicken β-actin promoter, the epo gene, an internal ribozyme entry site, the Bcl2 gene, and a polyA sequence.

Rag1/B16 mice were infused with 5×10¹¹ copies of the rAAV. Approximately 5% of the hepatocytes were found to express Bcl2, as detected by immunofluorescence staining, and serum epo concentration was found to reach 2000 IU/ml at 4 weeks post viral administration.

All documents cited herein are incorporated by reference. Numerous modifications and variations of the present invention are included in the above-identified specification and are expected to be obvious to one of skill in the art. Such modifications and alterations to the compositions and processes of the present invention are believed to be encompassed in the scope of the claims appended hereto.

What is claimed is:

1. A method for gene transfer into a population of cells capable of regeneration comprising the steps of:
(a) exposing a population of host cells to a recombinant viral vector capable of replication during cellular division comprising a gene encoding an anti-apoptotic agent, a selected transgene, and regulatory sequences which control expression of said anti-apoptotic agent and said transgene, whereby at least a subpopulation of said host cells is infected with said recombinant viral vector;
(b) contacting said population of host cells with an apoptotic agent, whereby said subpopulation of infected host cells which express said anti-apoptotic agent and the product of said transgene are protected against apoptosis; and
(c) allowing said protected cells to replicate.

2. The method according to claim 1, wherein said anti-apoptotic agent is an anti-apoptotic member of the Bcl2 family.

3. The method according to claim 1, wherein said anti-apoptotic agent is Bcl2.

4. The method according to claim 1, wherein said apoptotic agent is selected from the group consisting of non-neutralizing anti-fas antibodies and tumor necrosis factor.

5. The method according to claim 1, wherein said exposing step (a) comprises administering said recombinant viral vector to said host cells at a dose of about 1×10⁸ to about 1×10¹³ plaque forming units.

6. The method according to claim 1, wherein said recombinant viral vector is an adeno-associated viral vector.

7. The method according to claim 1, wherein said transgene is selected from the group consisting of low density lipoprotein receptor, very low density lipoprotein receptor, growth hormone, Factor IX, ornithine transcarbamylase, carbamyl phosphate synthetase, arginino-succinate lysase, arginase, and arginino-succinate synthetase.

8. The method according to claim 1, wherein said host cells are hepatocytes.

9. A method for gene transfer comprising the steps of:
(a) exposing a population of host cells to a first recombinant viral vector capable of replicating upon cell division comprising a gene encoding an anti-apoptotic agent and regulatory sequences which control expression thereof, whereby at least a subpopulation of said host cells is infected with said first recombinant viral vector and expressed said anti-apoptotic agent;
(b) exposing said population of host cells to a second recombinant viral vector capable of replicating upon cell division comprising a selected transgene and regulatory sequences which control expression thereof, whereby at least a subpopulation of said host cells are infected with said second recombinant viral vector and express the product of said transgene; and
(c) contacting said population of host cells with an apoptotic agent, whereby said subpopulation of host cells which express said anti-apoptotic agent are protected against apoptosis

10. The method according to claim 9, wherein said anti-apoptotic agent is selected an anti-apoptotic member of the Bcl2 family.

11. The method according to claim 9, wherein said anti-apoptotic agent is Bcl2.

12. The method according to claim 9, wherein said apoptotic agent is selected from the group consisting of non-neutralizing anti-fas antibodies and tumor necrosis factor.

13. The method according to claim 9, wherein said exposing steps (a) and (b) comprise administering said first and second recombinant viral vectors to said host cells at a dose of about 1×10⁸ to about 1×10¹³ plaque forming units.

14. The method according to claim 9, wherein said first recombinant viral vector is an adeno-associated viral vector.

15. The method according to claim 9, wherein said second recombinant viral vector is selected from the group consisting of adenoviral vectors, hybrid adenovirus/adeno-associated viral vectors, and retroviral vectors.

16. The method according to claim 9, wherein said transgene is selected from the group consisting of low density lipoprotein receptor, very low density lipoprotein receptor, growth hormone, Factor IX, ornithine transcarbamylase, carbamyl phosphate synthetase, arginino-succinate lysase, arginase, and arginino-succinate synthetase.

17. The method according to claim 9, wherein said host cells are hepatocytes.

18. A recombinant viral vector which replicates during division of a host cell for use in gene transfer comprising a gene encoding an anti-apoptotic agent, a selected transgene, and regulatory sequences which direct expression of the anti-apoptotic agent and the transgene product.

19. The recombinant viral vector according to claim 18, wherein said anti-apoptotic agent is Bcl2.

20. The recombinant viral vector according to claim 18 which is an adeno-associated viral vector.

21. A pharmaceutical composition comprising the recombinant viral vector according to claim 20.
